SEQUENCE LISTING (10 sequences)

SEQ ID NO: 1 - DNA, 40 bp, synthetic construct
  misc_feature 1..40: note = synthetic primer
  source 1..40: mol_type = other DNA
cacacggtctctcatcgataaggcgtttccatccgtcacg

SEQ ID NO: 2 - DNA, 37 bp, synthetic construct
  misc_feature 1..37: note = synthetic primer
  source 1..37: mol_type = other DNA
cacacggtctcagatggcgcctctgaacaaggtcacc

SEQ ID NO: 3 - DNA, 39 bp, synthetic construct
  misc_feature 1..39: note = synthetic primer
  source 1..39: mol_type = other DNA
cacacggtctcagatggactacaactgccacacaccacc

SEQ ID NO: 4 - DNA, 26 bp, synthetic construct
  misc_feature 1..26: note = synthetic oligomer
  source 1..26: mol_type = other DNA
agctgtccacctacgagaacctgtcc

SEQ ID NO: 5 - DNA, 39 bp, synthetic construct
  misc_feature 1..39: note = synthetic oligomer
  source 1..39: mol_type = other DNA
tttttttttttttttttggacaggttctcgtaggtggac

SEQ ID NO: 6 - DNA, 33 bp, synthetic construct
  misc_feature 1..33: note = synthetic oligomer
  source 1..33: mol_type = other DNA
tctcttctctctggcaggtgaaggactccttcg

SEQ ID NO: 7 - DNA, 25 bp, synthetic construct
  misc_feature 1..25: note = synthetic oligomer
  source 1..25: mol_type = other DNA
gatgcgaaggagtccttcacctgcc

SEQ ID NO: 8 - DNA, 24 bp, synthetic construct
  misc_feature 1..24: note = synthetic oligomer
  source 1..24: mol_type = other DNA
cgggatggcggaagatatgcagag

SEQ ID NO: 9 - DNA, 28 bp, synthetic construct
  misc_feature 1..28: note = synthetic oligomer
  source 1..28: mol_type = other DNA
catcctctgcatatcttccgccatcccg

SEQ ID NO: 10 - DNA, 13 bp, synthetic construct
  misc_feature 1..13: note = synthetic oligomer
  source 1..13: mol_type = other DNA
ttcagagtctgac

(19) United States
(12) Patent Application Publication
(10) Pub. No.: US 2025/0019743 A1
(43) Pub. Date: Jan. 16, 2025

(54) METHOD TO PRODUCE DNA MOLECULES WITH REPEATING UNITS FOR USE IN SINGLE-MOLECULE ASSAYS

(71) Applicant: LUMICKS DSM HOLDING B.V., Amsterdam (NL)

(72) Inventor: Marco SIMONETTA, Amsterdam (NL)

(73) Assignee: LUMICKS DSM HOLDING B.V., Amsterdam (NL)

(21) Appl. No.: 18/711,578

(22) PCT Filed: Nov. 21, 2022
(86) PCT No.: PCT/NL2022/050667

§ 371 Date: May 18, 2024

(30) Foreign Application Priority Data
Nov. 22, 2021 (NL) 2029859

(51) Int. Cl. C12Q 1/6806

(52) U.S. Cl. CPC C12Q 1/6806

(57) ABSTRACT

The invention relates to a method for producing linear DNA molecules with at least two or more repeating units of a target DNA sequence for use as DNA substrate in single-molecule assays, wherein a target DNA is subjected to a ligation reaction and the ligation reaction is stopped when the reaction reaches the end of the linear phase to block circularization of the DNA molecules. Compositions comprising such linear DNA molecules and use of such compositions in single-molecule assays are also provided.

The invention relates to a method for producing linear DNA molecules with at least two or more repeating units of a DNA sequence for use as DNA molecule in single-molecule assays, compositions comprising such linear DNA molecules and use of such compositions in single-molecule assays.

Single-molecule methods have been developed to study the dynamics of molecular processes in real-time. By monitoring the time evolution of single molecules as they undergo mechanical or (bio)chemical processes detailed information about the underlying mechanism of these processes can be obtained.

Optical tweezers are scientific tools that use a highly focused laser beam to trap and manipulate microscopic or nanoscopic objects that are attached between polymer or glass beads, which are often functionalized beads to facilitate specific attachment of the microscopic or nanoscopic objects. By changing the focus position of the laser beam the microscopic or nanoscopic objects may be moved around at will in a similar way as trapping an object between tweezers. Optical tweezers are sometimes also referred to as optical traps. Microscopic or nanoscopic objects such as for example cells or molecules may be attached to such beads to allow manipulation of the microscopic or nanoscopic objects. For example, a molecule may be attached on one side to a surface (e.g., the surface of a microscope cover slide) and on the other side to a microscopic bead. Manipulation of the position of the optical trap (and thus the bead) can then allow for example stretching of the molecule. If two optical trapping beams are provided molecules or other microscopic or nanoscopic objects can be tethered between two functionalized beads. This allows stretching of such objects by changing the distance between the optical traps. The polymer or glass beads are generally between 100 nm and 10 μm. Molecules are typically between a few 100 nm and a few tens of micrometres.

In biology and medicine, optical tweezers can be used to trap a single bacterium, a cell or a nanomolecule such as a DNA molecule to study mechanistic properties of these objects, or the mechanism underlying the biochemical processes these objects are involved in. Optical tweezers have been proven especially useful in real time analysis of protein binding to DNA molecules trapped between the two functionalized beads. A DNA molecule trapped between two functionalized beads is also referred to as a DNA tether.

Proteins binding to DNA can for example be studied using optical techniques. For example, they can be imaged using (single-molecule) fluorescence imaging techniques. E.g., by labelling proteins with fluorescent or luminescent moieties and providing excitation light and sensitive photodetectors one can detect and track single proteins (or other molecules) on DNA strands. See for example Candelli, A., Wuite, G. J. L., & Peterman, E. J. G. (2011). Combining optical trapping, fluorescence microscopy and micro-fluidics for single molecule studies of DNA—protein interactions, 7263-7272 (https://doi.org/10.1039/c0cp02844d) and Heller, I., Sitters, G., Broekmans, O. D., Farge, G., Menges, C., Wende, W., . . . Wuite, G. J. L. (2013). STED nanoscopy combined with optical tweezers reveals protein dynamics on densely covered DNA. Nature Methods, 10(9), 910-916 (https://doi.org/10.1038/nmeth.2599). Other examples of methods for imaging and or localizing objects or molecules binding to DNA are inverse Binding-Activated Localization Microscopy (iBALM) (WO 2019/151864A8, Meijering, A. E. C., Biebricher, A. S., Sitters, G., Brouwer, I., Peterman, J. G., Wuite, G. J. L., & Heller, I. (2020). Imaging unlabeled proteins on DNA with super-resolution. Nucleic Acids Research, 48(6) (https://doi.org/10.1093/nar/gkaa061), interferometric scattering microscopy (iSCAT) (Ortega-Arroyo, J., & Kukura, P. (2012). Interferometric scattering microscopy (iSCAT): new frontiers in ultrafast and ultrasensitive optical microscopy. Physical Chemistry Chemical Physics, 14(45), 15625 (https://doi.org/10.1039/c2cp41013c) or back-focal plane interferometry (WO 2019/093895A1).

Quantitative characterization of protein binding to a target DNA sequence requires the analysis of many protein binding events. DNA tethers with multiple protein binding sites will accelerate generation of data required for the quantitative analysis. Such DNA tethers can be obtained by trapping a linear DNA molecule comprising multiple copies of a target DNA sequence arranged in tandem.

Methods to produce DNA molecules comprising multiple copies of a DNA sequence of interest are known in the art. A problem often encountered in the production of DNA molecules comprising multiple copies of a target DNA sequence arranged in tandem is the intramolecular circularization that occurs during ligation of the target DNA sequence. During ligation, the target DNA will be joined together to form DNA molecules varying in length and number of repeating units of the target DNA sequence. As the ligation reaction progresses, ligation occurs not only between two different DNA molecules but also intramolecularly between the two ends within a DNA molecule, resulting in circular DNA molecules. At the end of the ligation reaction, most of the target DNA will have been joined together into circular DNA molecules varying in length and number of repeating units of the target DNA sequence. While the use of a circular DNA molecule may suffice as DNA substrate for some single-molecule assays, for dual beam optical tweezer methods the DNA molecule must be linear. Hence, there is a need for a process that produces DNA molecules with two or more repeating units of a target DNA sequence, wherein at least part of these DNA molecules is linear.

It was surprisingly found that linear DNA molecules comprising at least two or more repeating units of a DNA target sequence can be obtained by subjecting a target DNA sequence to a ligation reaction in the presence of a DNA ligase to obtain a first mixture of DNA molecules varying in length and number of repeats, wherein each DNA molecule comprises at least two or more repeating units of the target sequence and stopping the ligation reaction when the reaction reaches the end of the linear phase.

The ligation reaction is a reaction in which two strands of DNA are joined together by a DNA ligase which catalyzes the formation of a phosphodiester bond between the 3′hydroxyl group at the end of one DNA strand and the 5′phosphate group of another DNA strand. The ligation reaction is characterized in the beginning by a linear phase during which strands of DNA are rapidly joined together to form linear DNA strands of different length. However, when the ligation reaction reaches completion, most of the DNA molecules that were obtained were found to be circularized. This circularization results from the DNA ligase not only catalyzing intermolecular ligation between two strands of DNA but also intramolecular ligation between the 5′phosphate and the 3′hydroxyl of the same molecule. Thus, the ligation reaction is a balance between intermolecular and intramolecular ligation. When the amount of free 5′phosphate and 3′hydroxyl is high, the ligation is predominantly between two different DNA molecules. When the amount of free 5′phosphate and 3′hydroxyl is low, the ligation is predominantly intramolecularly between the ends of the same molecule. Without being bound by theory it was found that the shift from ligation being predominantly intermolecular between two DNA molecules to ligation being predominantly intramolecular between the ends of the DNA molecule is characterized by a saturation in growth of the length of the DNA molecules. Thus, the end of the linear phase of the ligation reaction is defined as the point in time where the production of new linear DNA molecules starts to slow down until few to no new linear DNA molecules are formed and intramolecular ligation (i.e., circularization) becomes predominant.

Stopping the ligation reaction at the end of the linear phase of the reaction allows sufficient production of DNA molecules of sufficient length and number of repeats without all DNA molecules being circularized. Without being bound by theory it was found that the end of the linear phase of the ligation reaction provided an optimum between the amount of linear DNA molecules still present in the reaction mixture and length and number of repeats in these linear DNA molecules.

Thus, the invention provides for a method for producing linear DNA molecules comprising at least two or more repeating units of a target sequence, comprising the steps of:
a. providing a target DNA sequence and a DNA ligase and subjecting the target DNA sequence to a ligation reaction to obtain a first mixture of DNA molecules varying in length and number of repeats, wherein each DNA molecule comprises at least two or more repeating units of the target sequence,
b. stopping the ligation reaction at the end of the linear phase of the ligation reaction, and
c. recovering said first mixture of DNA molecules.

The target DNA sequence that can be used in the method of the invention preferably is a double-stranded DNA molecule comprising a DNA sequence of interest, more preferably a double stranded DNA molecule having a DNA sequence of interest. The target DNA sequence (hereinafter referred to as target DNA) preferably has a size in the range of, and including, 50 to 5000 base pairs (bp), preferably 400 to 4000 bp, more preferably 1500 to 3500 bp. Highly preferred is a target DNA with a size in the range of, and including, 2000 to 3000 bp.

The size of the target DNA sequence generally depends on the single-molecule technique that will be used to study the dynamics of the DNA sequence of interest. In the event optical techniques are used to study the behaviour or dynamics of objects or molecules binding to the DNA sequence of interest, it may be advantageous to ensure that tandem repeats of the DNA sequence of interest are spaced by more than the optical point spread function of the microscope used to study the interaction. For example, in an optical tweezers system using 488 nm excitation light the fluorescent point spread function may be on the order of 250 nm FWHM. In that case the preferred spacing between binding sites may be more than 250 nm (corresponding to 735 bp at around 10 pN tension to approximately 430 bp in overstretched DNA) to ensure binding events on binding sites in neighbouring repeat units may be easily differentiated. More preferably the binding sites may be spaced by more than 500 nm, which corresponds to 1470 bp at 10 pN and 860 bp in overstretched DNA.

Preferably, the concentration of the target DNA sequence in the ligation reaction according to the invention has a concentration in the range of, and including, 5 nM-1 μM, preferably in the range of, and including, 25 to 250 nM.

The target sequence can be obtained by PCR amplification of the DNA sequence of interest using primers comprising a recognition site of a restriction enzyme and subjecting the amplified DNA sequence to digestion with said restriction enzyme to form the target DNA sequence. The target sequence can also be obtained from a plasmid comprising the DNA sequence of interest and digesting the plasmid with a restriction enzyme after amplification of the plasmid in a host cell. Amplification of a DNA sequence of interest and digestion of the amplified DNA sequence with restriction enzymes can be carried out using routine skills and standard cloning and recombinant DNA techniques commonly known in the art. Alternatively, the target DNA sequence can also be produced using standard techniques of artificial gene synthesis.

Preferably, amplification and digestion, i.e. cleavage of the DNA by a restriction enzyme, of the DNA sequence of interest is carried out to provide a target DNA sequence with complementary protruding single-stranded overhangs at both ends (hereinafter referred to as sticky ends). Preferably, the restriction enzymes are restriction enzymes that produce complementary single-stranded overhangs at the ends after cleavage. More preferably, the restriction enzymes are Type II restriction enzymes, preferably restriction enzymes selected from the group of Type IIP and Type IIS restriction enzymes.

The ligation reaction of the target DNA sequence can be directional or non-directional, depending on the restriction enzyme that is selected to produce the target DNA sequence. If the cleavage site of the selected restriction is palindromic, the resulting complementary single stranded overhangs at the ends of the target DNA sequence will allow not only annealing of the overhangs in a head-to-tail direction, but also in a head-to-head and tail-to-tail direction as shown in FIG. 1A. If the cleavage site of the selected restriction enzyme is not-palindromic, annealing of the overhangs is only allowed in a head-to-tail fashion as shown in FIG. 1B. Thus, selection of the type of restriction enzyme in the formation of the target DNA sequence will determine the directionality of the ligation.

The ligation reaction is carried out in the presence of a DNA ligase using standard ligation techniques known in the art. DNA ligation kits comprising a suitable DNA ligase and the necessary buffer compositions and co-factors required for the ligation reaction are commercially available. Suitable DNA ligases are T4, T7 and E. coli DNA ligases. Preferably, the DNA ligase is a T4 DNA ligase.

The ligation reaction is generally carried out at a temperature recommended by the manufacturer of the commercial kit. As a guideline, the temperature can be in the range of, and including, 4-37° C., preferably 10-25° C. Suitable ligation results have been obtained with temperatures of 12, 14, 16, 18, 20, 22, or 24° C., in particular with temperatures of 18, 20 and 22° C.

The end of the linear phase of the ligation reaction can be determined for any given amount of target DNA sequence and DNA ligase by carrying out a time course assay of the intended ligation reaction, wherein the target DNA and DNA ligase are brought under the same reaction conditions under which the intended ligation reaction is to be carried out, and samples of the reaction mixture are taken at predetermined points in time. A time course assay involves taking samples at different points in time to establish the course of the reaction and determine the end of the linear phase of the ligation reaction (FIGS. 3A and 3B). A time course assay may also involve testing different concentrations of target DNA and DNA ligase respectively to find which concentrations give the best results i.e., the highest number of repeats. Analyzing the content of the samples for yield and size of resulting DNA molecules by agarose gel electrophoresis allows the determination of the end of the linear phase of the ligation reaction. Plotting the production rate in time provides a calibration graph. See for example FIG. 1C.

The ligation reaction can be stopped at the end of the linear phase of the ligation reaction by the addition of a ligase inhibitor such as EDTA and the like to the reaction mixture, or by heating the reaction mixture to a temperature in the range of 65-70° C. to denature the DNA ligase. After the ligation is stopped, the first mixture of DNA is recovered to provide a first mixture of DNA molecules varying in length and number of repeats, wherein each DNA molecule comprises at least two or more repeating units of the target sequence, and wherein part of the DNA molecules consists of linear DNA molecules. With the method of the invention, linear DNA molecules comprising at least two or more repeating units of the target DNA, preferably at least 3, 4, 5, 6, 7, 8, 9, 10, or more repeating units of the target DNA could be obtained. If the ligation reaction is non-directional, linear DNA molecules comprising up to 15 or more repeating units of the target DNA can be obtained, preferably even up to 16, 17, 18, 19 or 20 or more repeating units of the target DNA.

After the ligation has been stopped, the first mixture of DNA molecules comprising the linear DNA molecules can be recovered. After recovery, the first mixture of DNA molecules is subjected to a second ligation step in the presence of a DNA ligase and DNA adaptors. If the ligation has been stopped by the addition of a ligase inhibitor, the first reaction mixture of DNA molecules must be purified prior to subjecting the mixture to the second ligation step to remove the inhibitor. This purification can be carried out using standard DNA purification methods.

The DNA adaptors of the present invention are short double-stranded DNA molecules that lack a free 5′phosphate at one end and comprise at the other end protruding single-stranded overhangs which are complementary to the single-stranded overhangs of the linear DNA molecules present in the first mixture of DNA molecules. The absence of a free 5′phosphate prevents that end of the DNA adaptor from being attached to another DNA molecule by the DNA ligase. Preferably, the DNA adaptors according to the invention are double-stranded DNA molecules having a size in the range of 10-50 bp, preferably 15-35 bp, more preferably 18-30 bp.

Preferably, the initial concentration of the DNA adaptors according to the invention in the second ligation reaction is in the range of, and including, 5-3000 nM, preferably 25-250 nM to create at least a five-fold excess compared to the concentration of DNA molecules.

The DNA adaptors can be obtained by synthesizing two complementary single-stranded DNA oligomers and annealing the two complementary using routine skills and standard techniques.

Adaptors lacking a free 5′phosphate at one end can be obtained by de-phosphorylation of the 5′phosphate, by synthesis without the 5′ phosphate directly or by reacting that 5′phosphate with a blocking moiety.

The second ligation step allows the free single-stranded overhangs at both ends of the linear DNA molecules to anneal to the complementary single-stranded overhang of the DNA adaptor, so that the linear DNA molecules present in the first mixture of DNA molecules become attached between two adaptor molecules, hereinafter referred to as adapted linear DNA molecules (FIGS. 2A and 2B). Thus, by subjecting the first mixture of DNA molecules obtained with the method of the invention to a second ligation reaction, the invention further provides for a second mixture of DNA molecules that vary in length and number of repeats, wherein each DNA molecule comprises at least two or more repeating units of the target DNA, and wherein part of the DNA molecules consists of adapted linear DNA molecules.

The second mixture of DNA molecules comprising the adapted linear DNA molecules can be subjected to a functionalization step wherein one or more functional moieties are attached to both ends of the adapted linear DNA molecules. Suitable functional moieties are moieties that allow binding to functionalized beads of the optical tweezers. Preferably the functional moiety attached to the ends of the functionalized linear DNA molecules is one half of a conjugation pair such as biotin-streptavidin pair or digoxigenin-anti-digoxigenin-antibody pair. More preferably the functional moiety is selected from the group of biotins and digoxigenins.

The adapted linear DNA molecules can be labeled with one or more functional moieties by subjecting the adapted linear DNA molecule to a standard enzymatical or chemical reactions in the presence of a mixture of nucleotides and nucleotides covalently attached to the functional moiety. For example, a klenow reaction is a well-known reaction to label DNA with biotin moieties and can be carried out using standard techniques. Alternatively, the adapted linear DNA molecules can be functionalized by attaching a single-stranded oligomer, which has one or more of the functional moieties attached thereto, to the ends of the DNA molecule using standard techniques. Thus, by subjecting the second mixture of DNA substrates obtained with the method of the invention to a functionalization step, a mixture of DNA molecules varying in length and number of repeats could be obtained, wherein each DNA molecule in the mixture comprises at least two or more repeating units of the target sequence, and wherein part of the DNA molecules consists of functionalized linear DNA molecules. Preferably, the functionalized linear DNA substrates are linear DNA molecules labeled with biotin or digoxigenin. The functionalized linear DNA molecules may also be functionalized with two different functional moieties, for example one or more biotin moieties attached at one end of the linear DNA molecule and one or more digoxigenin moieties attached to the other end. The method of the invention provides for a mixture of DNA molecules varying in length and number of repeats, wherein each DNA molecule comprises at least two or more repeating units of the target sequence, and wherein part of the DNA molecules consists of functionalized linear DNA molecules.

In an alternative method of the invention, after stopping the ligation reaction, the first mixture of DNA molecules obtained after recovery may be subjected directly to a functionalization step wherein one or more functional moieties are attached to both ends of the linear DNA molecules that are present in the mixture.

The DNA molecules of the first mixture may be functionalized by attaching a single-stranded oligomer, which has one or more functional moieties attached thereto, to the ends of the DNA molecule using standard techniques. The DNA molecules of the first mixture may also be functionalized by subjecting the adapted linear DNA molecule to a standard enzymatical or chemical reactions in the presence of a mixture of nucleotides and nucleotides covalently attached to the functional moiety. For example, a klenow reaction is a well-known reaction to label DNA with biotin moieties and can be carried out using standard techniques.

Thus, by subjecting the first mixture of DNA substrates obtained with the method of the invention directly to a functionalization step, a mixture of DNA molecules varying in length and number of repeats could be produced, wherein each DNA molecule in the mixture comprises at least two or more repeating units of the target sequence, and wherein part of the DNA molecules consists of functionalized linear DNA molecules.

In a preferred embodiment of the invention, the DNA adaptors have been functionalized with one or more functional moieties prior to addition in the second ligation step. The DNA adaptors can be labeled with one or more functional moieties by subjecting the DNA adaptor to a standard enzymatical or chemical reaction in the presence of a mixture of nucleotides and nucleotides covalently attached to the functional moiety. Examples of such nucleotides attached to a functional moiety are dT labeled with biotin or dT labeled with digoxigenin. The klenow reaction, for example, is a well-known reaction that can be carried out using standard techniques to label DNA with biotin moieties.

Functionalized DNA adaptors can also be prepared by synthesizing and annealing two complementary single-stranded DNA oligomers, wherein the 5′end of one of the single stranded DNA oligomers comprises a string of nucleotides comprising one or more nucleotides covalently attached to a functional moiety. The complementary single-stranded DNA oligomers are so designed that annealing of the two strands produce a DNA adaptor comprising a single-stranded overhang with one or more functional moieties at one end of the adaptor molecule and protruding single-stranded overhangs that are complementary to the free single-stranded overhangs of the linear DNA molecules at the other end.

Thus, in a preferred embodiment, the invention provides for a method wherein the DNA adaptors that are added in the second ligation step have been functionalized with a functional moiety prior to addition to the mixture of DNA molecules. Ligation with functionalized DNA adaptor molecules allow attachment of the functionalized DNA adaptor molecule to both ends of the linear DNA molecules that are present in the first mixture of DNA molecules resulting in the formation of functionalized linear DNA molecules of the invention (FIGS. 2A and 2B). Subjecting the first mixture of DNA substrates to a second ligation step in the presence of functionalized DNA adaptor molecules produces a second mixture of DNA molecules varying in length and number of repeats, wherein each DNA molecule comprises at least two or more repeating units of the target sequence, and wherein part of the DNA molecules consists of functionalized linear DNA molecules. This method has the advantage, that the addition of functionalized DNA adaptors in the second ligation step obviates the need for a separate functionalization step of the linear DNA molecules, thereby simplifying the production of functionalized linear DNA molecules comprising at least two or more repeats of a target DNA sequence arranged in tandem that are suitable for use in a single-molecule assay.

Preferably, the initial concentration of the functionalized DNA adaptors according to the invention at the start of the second ligation reaction is in the range of, and including, 300-3000 nM, to create at least a five-fold excess compared to the concentration of DNA molecules.

The target DNA sequence, the DNA ligase, and the DNA adaptor molecules that can be used in any of the methods of the invention can be used and prepared as described herein before.

In an even more preferred embodiment of the invention, the DNA adaptor molecules are added in the first ligation step when the ligation reaction reaches the end of the linear phase to allow the formation of adapted linear DNA molecules, i.e. linear DNA molecules that are attached at both ends to the DNA adaptor molecules. It was found that the addition of DNA adaptor molecules in the first ligation step of the method of the invention also blocks intramolecular circularization of the remaining linear DNA molecules (See FIG. 5A). The addition of the DNA adaptor molecules in the first ligation step has the advantage of obviating a second ligation step, thereby allowing ligation of the target DNA sequence to obtain DNA substrates comprising at least two or more repeating units of target DNA sequence and ligation of the DNA adaptor molecules to said linear DNA molecules to be carried out in one single ligation step to produce a reaction mixture of DNA molecules varying in length and number of repeats, wherein each DNA molecule comprises at least two or more repeating units of the target sequence, and wherein part of the DNA molecules consist of adapted linear DNA molecules.

Thus, the invention also provides for a method for producing linear DNA molecules with two or more repeating units of a target sequence, comprising the steps of:
a. providing a target DNA sequence and a DNA ligase and subjecting the target DNA sequence to a ligation reaction to produce a first mixture of DNA molecules varying in length and number of repeats, wherein each DNA molecule comprises at least two or more repeating units of the target sequence,
b. adding DNA adaptor molecules at the end of the linear phase of the ligation reaction of step a) to produce a second mixture of DNA molecules varying in length and number of repeats, wherein each DNA molecule comprises at least two or more repeating units of the target sequence, and wherein part of the DNA molecules consists of adapted linear DNA molecules.

The mixture of DNA molecules obtained in step b) can then be subjected to a functionalization step as described here before to produce the mixture of DNA molecules varying in length and number of repeats, wherein each DNA molecule comprises at least two or more repeating units of the target sequence, and wherein part of the DNA molecules consists of functionalized linear DNA molecules. The target DNA sequence, the DNA ligase, the DNA adaptor molecules that can be used in any of the methods of the invention can be prepared and used as described herein before.

In another preferred embodiment of the invention, functionalized DNA adaptor molecules are added in the first ligation step when the ligation reaction reaches the end of the linear phase to allow the formation of functionalized linear DNA molecules, i.e. linear DNA molecules that are attached to at both ends to the functionalized DNA adaptor molecules. The addition of functionalized DNA adaptor molecules in the first ligation step not only blocks intramolecular circularization of the remaining linear DNA molecules, but at the same functionalizes the linear DNA molecules that are produced during the ligation reaction. Thus, the invention also provides for a method for producing linear DNA molecules with two or more repeating units of a target sequence, comprising the steps of:
a. providing a target DNA sequence and a DNA ligase and subjecting the target DNA sequence to a ligation reaction to produce a first mixture of DNA molecules varying in length and number of repeats, wherein each DNA molecule comprises at least two or more repeating units of the target sequence, and
b. adding functionalized DNA adaptor molecules at the end of the linear phase of the ligation reaction to produce a second mixture of DNA molecules varying in length and number of repeats, wherein each DNA molecule comprises at least two or more repeating units of the target sequence, and wherein part of the DNA molecules consists of functionalized linear DNA molecules.

This method not only obviates a separate second ligation step, but also obviates a separate functionalization step, thereby reducing the number of steps that must be carried out to produce a mixture of DNA molecules varying in length and number of repeats, wherein each DNA molecule comprises at least two or more repeating units of the target sequence, and wherein part of the DNA molecules consists of functionalized linear DNA molecules.

A further advantage is that ligation of the target DNA sequence to produce linear DNA molecules comprising at least two or more repeating units of the target sequence and functionalization of the linear DNA molecules obtained with ligation can be carried out in a single ligation reaction, i.e. a one-pot-synthesis of a mixture of DNA molecules varying in length and number of repeats, wherein each DNA molecule comprises at least two or more repeating units of the target sequence, and wherein part of the DNA molecules consists of functionalized linear DNA molecules functionalized linear. When all remaining linear DNA substrates in the reaction mixture have been ligated to functionalized DNA adaptor molecules the ligation reaction stops.

The target DNA sequence, the DNA ligase, and the DNA adaptor molecules, which optionally can be functionalized, to be used in any of the methods of the invention can be used and prepared as described herein before. The target DNA that can be used in the method of the invention may also be modified prior to ligation to comprise one or more lesions and/or a fluorescent label. Alternatively, said one or more lesions and/or fluorescent labels can be introduced into the DNA molecules that are obtained with any of the methods of the invention.

The mixture of DNA molecules varying in length and number of repeats, wherein each DNA molecule comprises at least two or more repeating units of the target sequence, and wherein part of the DNA molecules consists of functionalized linear DNA molecules that can be obtained with the various methods of the invention is suitable for use in any single-molecule assay, in particular single-molecule assays that require the DNA substrate to be linear such as the optical tweezer methods. The mixture of DNA molecules varying in length and number of repeats, wherein each DNA molecule comprises at least two or more repeating units of the target sequence, and wherein part of the DNA molecules consists of functionalized linear DNA molecules may be subjected to a purification step to remove components that may interfere with the single-molecule assay. Purification can be carried out using DNA purification methods well-known in the art. Commercial kits for DNA purification are available and may be used for purification of any mixture of DNA substrate obtained with the various methods of the invention.

Thus, the invention also provides for a composition comprising a mixture of DNA substrates which differ in length and number of repeating units and in which part of the DNA molecules are linear, functionalized DNA molecules and each DNA molecule comprising at least two or more repeats of the target DNA sequence. Such a composition is suitable for use as DNA substrate in any single-molecule assay, in particular single-molecule assays that require the DNA substrate to be linear such as the optical tweezer methods.

The composition obtained with the method of the invention comprises at least 5% by weight functionalized linear DNA molecules varying in length and number of repeating units, wherein the percentage is based on the total weight of DNA substrates present in the composition. Preferably, the composition comprises at least 10%, 11%, 12%, 13%, 14%, 15%, 16%, 17%, 18%, 19%, 20% by weight of linear, functionalized DNA molecules according to the invention.

The composition obtained with the method of the invention is suitable for use in any single-molecule assay, in particular single-molecule assays that require the DNA substrate to be linear such as the optical tweezer methods or DNA curtain methods (Visnapuu, M. L., & Greene, E. C. (2009). Single-molecule imaging of DNA curtains reveals intrinsic energy landscapes for nucleosome deposition. Nature Structural and Molecular Biology, 16(10), 1056-1062 (https://doi.org/10.1038/nsmb.1655)). By producing linear functionalized DNA molecules varying in length and number of repeats, wherein each DNA molecule comprises at least two or more repeating units of the target DNA, the method of the invention allows for accelerated data acquisition of interaction of molecules with the target DNA sequence due to the presence of multiple copies of the target DNA sequence in the DNA molecules. Interactions that can be studied are for example the interaction of proteins involved in repair of lesions or damage in the target DNA sequence, the binding dynamics of (blocked or catalytically dead) restriction enzymes, or the binding dynamics of CAS9 to its specific binding location as a function of force.

Thus, the invention further provides for a single-molecule assay, comprising the steps of a) providing a composition comprising a mixture of DNA substrates that differ in length and number of repeating units of a target DNA sequence, wherein part of the DNA substrates are linear functionalized DNA substrates, b) bringing the composition into contact with beads that have been functionalized with groups that recognize and bind specifically with the functional moieties of the functionalized linear DNA molecules present in the composition, to form DNA tethers i.e. linear DNA substrates trapped between the two or more beads of the single-molecule method, c) subjecting the DNA tethers to distance analysis to select the DNA tethers that have a predetermined length, and, d) measuring interaction of molecules with the DNA tether.

Preferably, the single-molecule assay is an optical tweezer assay, wherein the functionalized beads are beads that have been functionalized to bind specifically to the functionalized linear DNA substrates to form DNA substrates trapped between two beads of the optical tweezer.

As will be evident to the skilled person, different embodiments of the present invention can be combined unless they are mutually exclusive.

The following examples will illustrate the practice of the present invention in some of the preferred embodiments. Other embodiments within the scope of the claims will be apparent to one skilled in the art.

### LEGEND OF FIGURES

FIG. 1. Schematics of non-directional tandem ligation (NTL) and directional tandem ligation (DTL).

A) A DNA unit with HindIII restriction sites used for NTL. Digestion with HindIII forms palindromic overhangs that allow tandem ligation of the DNA units in random direction.

B) A DNA unit with Bsal restriction sites used for DTL. Digestion of this DNA unit with Bsal forms non-palindromic overhangs that allow tandem ligation of the DNA units in one direction (head-to-tail only).

C) Model of a tandem ligation reaction that represents the linear phase and plateau of the reaction (left). Linear phase occurs with high concentration of free DNA ends and is characterized by intermolecular DNA ligation with predominantly formation of linear DNA molecules (upper right panel). When most of the DNA ends are ligated, the reaction reaches the plateau with intramolecular DNA ligation and predominantly formation of circular DNA molecules (lower right panel). DNA units are represented by grey sticks delimited by black marks.

FIG. 2. Schematics of labelled DNA adaptors and dumbbell configurations of NTL and DTL

A) Biotin DNA adaptor (BDA) is characterized by overhangs compatible with overhangs formed by HindIII digestion. BDA is ligated to the tandem DNA repeats formed in NTL at the end of the linear phase, blocking ligation and allowing tethering of the tandem DNA repeats between two streptavidin-coated beads trapped in optical tweezers. White circles represent the five biotin moieties attached to one end of BDA. Attachment of the biotin moieties to one end of the BDA result in the absence of a free 5′phosphate at that end, thereby blocking that end from ligation.

B) Two different adaptors are used to label tandem DNA repeats formed in DTL. The digoxigenin DNA adaptor (DDA) contains an overhang compatible with the overhang formed by the digestion of Bsal at the 5′ of a DNA unit. Biotin DNA adaptor (BDA_2) contains an overhang compatible with the overhang formed by the digestion of Bsal at the 3′ of a DNA unit. DDA and BDA_2 are ligated to the ends of tandem repeats formed in DTL at the end of the linear phase blocking ligation (due to the absence of free 5′ phosphate on the adapters) and allowing tethering of the tandem DNA repeats between an anti-digoxigenin bead and a streptavidin-coated bead of optical tweezers. Dark circles represent the 3 digoxigenin moieties of DDA, while the white circles the 3 biotin moieties of BDA_2.

FIG. 3. Time courses of NTL.

A) A 30-minutes time course of NTL is analysed by gel agarose electrophoresis. There is no DNA band pattern change between 5 minutes and 30 minutes indicating that no intermolecular ligation is occurring in this phase of the reaction (plateau) and that intramolecular ligation circularized all the DNA molecules within 5 minutes. In the first lane, circular pUC19 runs as a supercoiled compared to the linearized pUC19 in the second lane. The smallest DNA band visualized in each time point of NTL runs as the supercoiled circular pUC19 (first lane), indicating a small percentage of DNA molecules that are circularized without being incorporated in tandem repeats. DNA bands containing up to 50 kb DNA are visualized at each time point of NTL, indicating that tandem repeats of up to 20 linearized pUC19 units (second lane) are formed within 5 minutes of NTL.

B) A 120-seconds time course of NTL is analysed by agarose gel electrophoresis. Black arrows indicate DNA bands that disappear or becomes less intense during the time course indicating DNA molecules that are incorporated in tandem repeats. White arrows indicate newly formed larger DNA tandem repeats. The pattern of DNA bands remains almost identical in time points after 60 seconds, indicating the plateau of NTL.

FIG. 4. Schematics of a tightrope assay with circular tandem repeats.

A circular NTL product obtained with NTL performed by Kong et al. 2017 (Kong, M., Beckwitt, E. C., Springall, L., Kad, N. M., & Van Houten, B. (2017). Single-Molecule Methods for Nucleotide Excision Repair: Building a System to Watch Repair in Real Time. Methods in Enzymology (1st ed., Vol. 592). Elsevier Inc. (https://doi.org/10.1016/bs.mie.2017.03.027)). In the tandem ligase described by Kong et al., 50 ng/ul of DNA (2.03 kb) were incubated with 0.25 U/ul Ligase T4 for 30 minutes at 22° C. 0 and. In addition, Kong et al. used polyethylene glycol in the ligase reaction mix. In an attempt to reproduce Kong et al. ligation reaction, we performed a ligation reaction using the same incubation time (30 min), same concentration of ligase and DNA, and same ligase buffer supplemented with polyethylene glycol. While we obtained the same prominent DNA band of ˜30-50 kb shown by Kong et al., all the attempts to biotinylate the ends of the tandem repeats failed, indicating that the DNA products of the tandem ligation described in Kong et al. are circular. Nonetheless, these circular tandem repeats are suitable for DNA tightrope assays as negatively charged beads can bind anywhere in the DNA sequence.

FIG. 5. BDA is ligated to the ends of DNA repeats blocking further ligation reactions.

A) DNA agarose gel electrophoresis was used to analyse the products of an NTL reaction supplemented with 0.5 μM BDA after 1 minute from the start of the reaction. White arrows indicate bands that disappear in after 3 minutes in NTL blocked with 0.5 mM EDTA, indicating that BDA is ligated to the ends of tandem repeats and blocks further ligation reactions.

B) Concentrations of 25 nM, 62.5 nM and 125 nM BDA were added to NTL after 1 minute reaction. All these concentrations of BDA terminate ligation as the DNA band pattern is similar to the one using 0.5 μM BDA (panel A).

FIG. 6. Analysis of NTL in optical tweezers experiments using C-Trap.

A) Three representative force distance curves obtained by tethering biotinylated DNA tandem repeats formed in NTL supplemented with BDA. DNA tethers are stretched using forces up to 60 pN. The contour length of the DNA has been determined by fitting the force distance data with the inverted Odijk model from the python package Pylake. The contour length can be converted to the number of base pairs and the number of base pairs can be converted to the number of ligated DNA units. A representative fitting of the most left force distance curve is shown.

B) Histogram representing the frequency of the number of DNA repeats observed analysing a total of 27 tethers. About 33% of the tethers contains more than 16 DNA tandem repeats.

FIG. 7. Analysis of DTL in optical tweezers experiments using C-Trap.

A) A representative image of single DNA molecule produced by DTL. The DNA molecule is tethered between an anti-digoxigenin bead and a streptavidin coated bead in the optical tweezers of C-Trap and imaged by fluorescence confocal microscopy. The anti-digoxigenin bead shows strong fluorescence due to the tethering procedure that involves a preincubation of the DNA molecules with the anti-digoxigenin bead according to LUMICKS' standard protocols. Visualization of seven ATTO647N fluorophores indicates that the tether is made of seven DNA tandem repeats. Importantly, ATTO647N fluorophores are at the same distance from each other (3041 bp) indicating that the tandem repeats are ligated in one direction. Equidistant ATTO647N fluorophores were observed in 100% of the tethers imaged in C-Trap (data not shown). The lower panel shows a schematic of the confocal image of the seven DNA repeats.

B) Histogram representing the frequency of the number of DNA repeats observed analysing a total of 32 tethers. About 60% of the tethers contains more than seven DNA tandem repeats.

### EXAMPLES

### Non-Directional Tandem Ligation Vs Directional Tandem Ligation

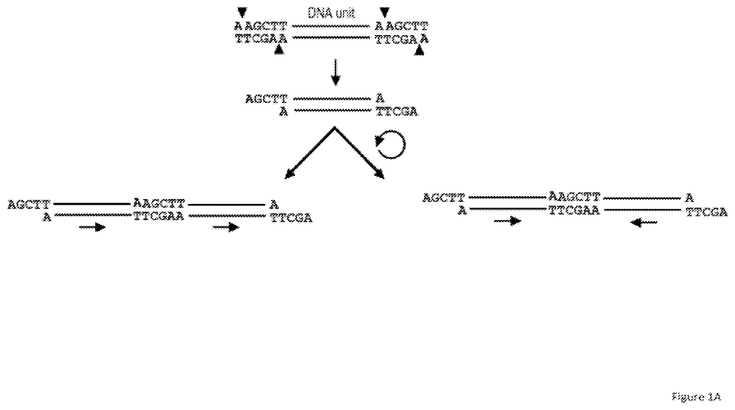

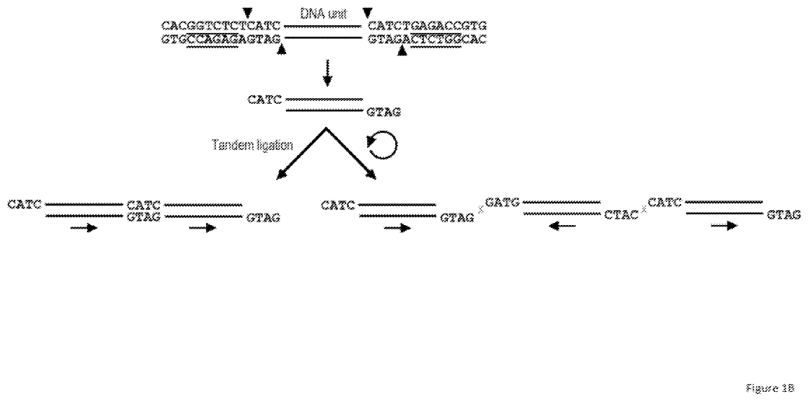

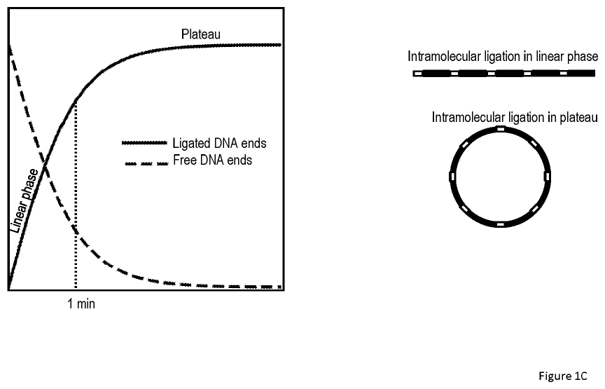

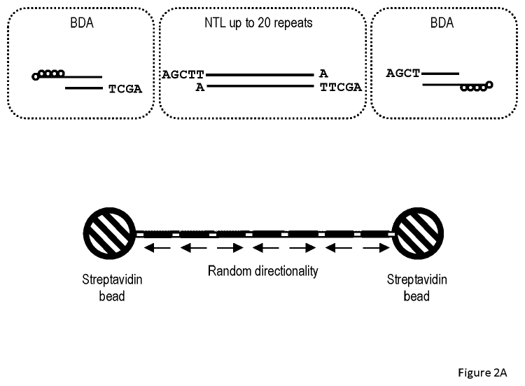

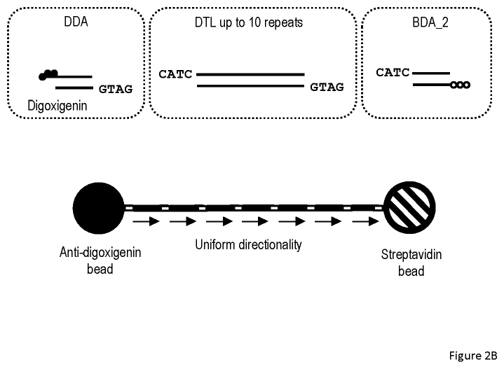

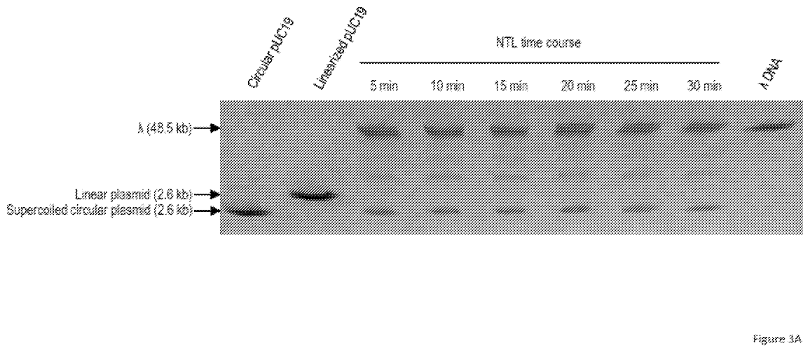

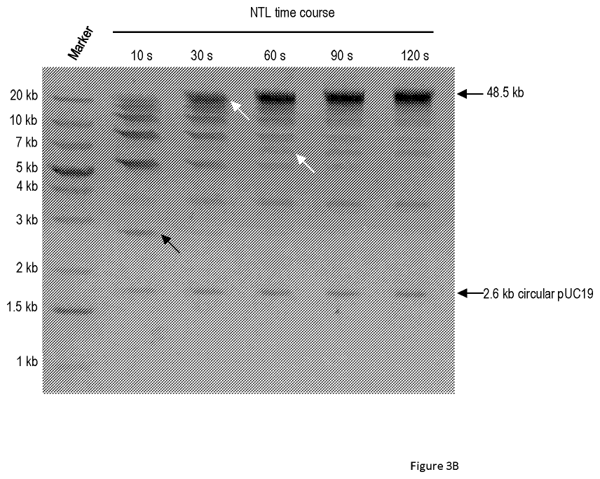

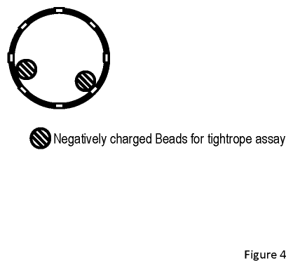

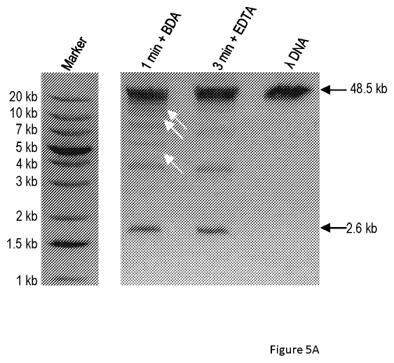

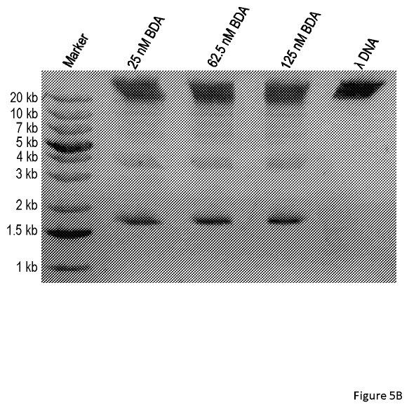

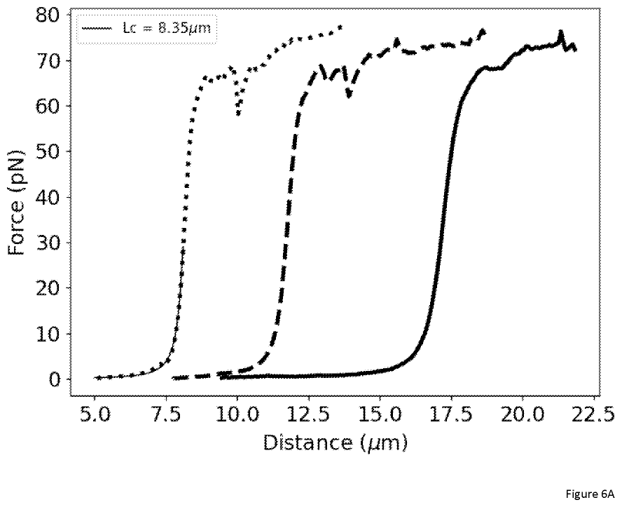

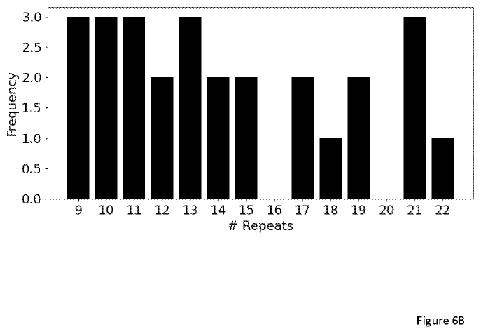

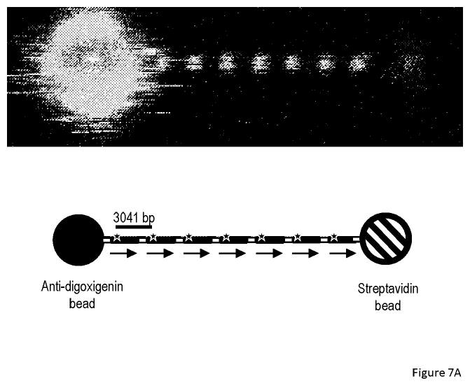

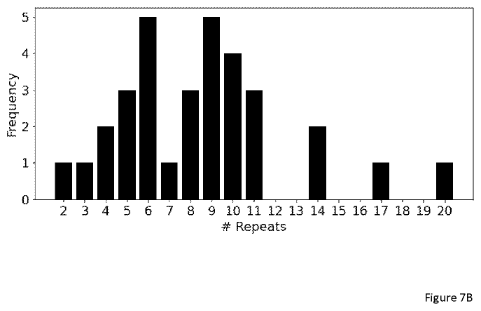

Non-directional tandem ligation uses DNA units digested with restriction enzymes that form DNA palindromic overhangs. As these overhangs are palindromic, the intermolecular ligation between DNA units can occur in random 5′-3′ direction (see FIG. 1A). Directional tandem ligation (DTL) uses Type IIS restriction enzymes that form non-palindromic overhangs allowing the intermolecular ligation in one 5′-3′ direction (see FIG. 1B). We only tested HindIII and Bsal in these methods. However, any restriction enzyme that forms palindromic DNA sequences can be used to prepare a target DNA sequence for NTL, while any Type IIS restriction enzymes can be used to prepare a target DNA sequence for DTL.

### Biotin DNA Adaptors to Label the Ends of Tandem Ligations for Tethering in Optical Tweezers

Both NTL and DTL, described in these methods, are characterized by a linear phase of ˜1 minute, during which high concentration of free DNA ends favours intermolecular ligation with fusion of multiple DNA repeats (see FIG. 1C). At the end of the linear phase, concentration of free DNA ends lowers, and intramolecular ligation becomes the main reaction. Most likely, a lower diffusion coefficient of the larger molecules formed during the linear phase of the reaction also contributes to increase the relative rate of intramolecular ligation over the intermolecular ligation. The resulting circular molecule formed at the end of tandem ligation reactions are not suitable for labelling of DNA ends and, thus, for tethering in the canonical dumbbell configuration of optical tweezers. Here, we use digoxigenin and biotin DNA adaptors that have compatible overhangs with the overhangs of the DNA tandem repeats (see FIGS. 2A and 2B). The addition of an excess of these DNA adaptors at the end of the linear phase of tandem ligation reactions, typically after 1 minute from the start of the reaction, leads to the quick ligation of these adaptors to the DNA ends. Biotin and digoxigenin moieties of adaptors attached to the ends of multiple DNA repeats block further ligation and thus the formation of circular DNA molecules by intramolecular ligation. Biotinylated and digoxigenin labelled DNA molecules can then be tethered to functionalized beads of optical tweezers (see FIGS. 2A and B).

TABLE 1
primer and adapter sequences. BT and DigT
refer to a thymidine residue
that is modified with respectively
biotin (B) or digoxigenin (Dig).

| | |
|---|---|
| Adaptor 1 | Phosphate-5′-AGCTGTCCACCTA |
| | CGAGAACCTGTCC-3′ |
| Adaptor 2 | Biotin-5′- |
| | T/TTT/BT/T/BT/TTT/BT/T/BT/ |
| | TTTTGGACAGGTTCTCGTAGGTGGA |
| | C-3′ |
| Fw Primer 3 | 5′-CACACGGTCTCTCATCGATAAGG |
| | CGTTTCCATCCGTCACG-3′ |
| Rv Primer 4 | 5′-CACACGGTCTCAGATGGCGCCTC |
| | TGAACAAGGTCACC-3′ |
| Rv Primer 5 | 5′-CACACGGTCTCAGATGGACTACA |
| | ACTGCCACACACCACC-3′ |
| Adaptor 3 | Digoxigenin-5′- |
| | T/CTC/DigT/TCTC/DigT/CTGGC |
| | AGGTGAAGGACTCCTTCG-3′ |
| Adaptor 4 | Phosphate-5′-GATGCGAAGGAGT |
| | CCTTCACCTGCC-3′ |
| Adaptor 5 | 3xBiotin-5′-CGGGATGGCGGAAG |
| | ATATGCAGAG-3′ |
| Adaptor 6 | Phosphate-5′-CATCCTCTGCATA |
| | TCTTCCGCCATCCCG |
| ATTO647N-13 nt | Phospate-5′-TTCAGAG/ |
| | (ATTO647N)-T/CTGAC |

### Example 1: Non-Directional Tandem Ligation

HindIII Digestion of pUC19

Non-directional tandem ligation (NTL) was performed using pUC19 plasmid (NEB, N3041) digested with HindIII restriction enzyme (NEB, R3104). pUC19 is 2686 bp and contains a unique HindIII restriction site, therefore HindIII digestion leads to the formation of a linear DNA molecule with HindIII overhangs. Linearized pUC19 plasmid is then purified using the PCR clean-up protocol of the DNA purification Promega Kit (Wizard® SV Gel and PCR Clean-Up System, A9281). Quality of HindIII digestion and purification was checked by agarose gel electrophoresis, which showed a unique DNA band corresponding to the expected size of 2686 bp linearized pUC19 (see FIG. 3A).

### Time Courses Identify Linear Phase of Non-Directional Tandem Ligation

Time courses of NTL was performed using 0.25 U/ul of Ligase T4 (Thermo Scientific™, 5 U/μL, EL001) and 50 ng/ul (57.4 nM) of linearized pUC19 in ligase buffer 1× (T4 DNA Ligase Buffer 10×, Thermo Scientific™ B69). These concentrations of Ligase T4 and linearized pUC19 have been used in every NTL described in these methods. Also, all NTL and DTL reactions occur at 22° C. Time courses are set up by pre-mixing DNA, Milli-Q and ligase buffer 10×. Then, the reaction is started by adding Ligase T4 and quickly mixing by pipetting. Aliquots are taken at each time point, quenched with 35 mM EDTA, and analysed by DNA agarose gel electrophoresis (FIG. 3A). A first time-course was performed over a 30-minutes NTL. After 5 minutes of reaction, no incorporation of short DNA molecules in longer DNA molecules is detected, indicating that ligation reaction reaches plateau within 5 minutes. A shorter time course of 120 seconds showed that the longest DNA molecules (20-50 kb) have already been formed after 30 seconds (FIG. 3B). Moreover, the high rate of intermolecular ligation (linear phase) is observed between 10 seconds and 60 seconds, while low rate is observed after 60 seconds (plateau), as shown in the reaction model in FIG. 1C. This was confirmed by unsuccessful attempts of biotinylating DNA molecules produced in NTLs longer that 5 minutes, while high efficiency of biotinylation is observed in NTLs of 1 minute (see below). Our 30 minutes long NTL is a reproduction of tandem ligation performed in Kong et al. 2017 (Kong, M., Beckwitt, E. C., Springall, L., Kad, N. M., & Van Houten, B. (2017). Single-Molecule Methods for Nucleotide Excision Repair: Building a System to Watch Repair in Real Time. Methods in Enzymology (1st ed., Vol. 592). Elsevier Inc. (https://doi.org/10.1016/bs.mie.2017.03.027)) indicating that DNA molecules with multiple repeats by Kong et al. 2017 are circular, and therefore non suitable for end labelling and tethering in optical tweezers. Negatively charged beads can interact internally to circular DNA molecules, therefore the circular molecules obtained in tandem ligation by Kong et al. 2017 are suitable for tightrope assay (see FIG. 4).

### Assembling Biotin DNA Adaptor (BDA)

BDA is formed upon annealing of two oligonucleotides (Table 1, Adaptor 1 and 2 and FIG. 2A) and is constituted by a 22 bp double stranded DNA formed by the complementary sequences of Adaptor 1 and 2 (Table 1, sequences in bold), flanked on one end with a 5′ overhang made of the 18 thymidines of the Adaptor 2 (Table 1, underlined sequence) and on the other end with a 5′ HindIII-compatible overhang of the Adaptor 1 (Table 1, underlined sequence). The 5′ of Adaptor 2 is modified with a biotin, which blocks the ligation reaction while allowing the binding to a streptavidin bead. Additional 4 biotins are attached internally to thymidines of the 18 thymidines overhang (Table 1, BT of Adaptor 2), to form additional binding sites with the streptavidin bead and stabilizes the tethers in optical tweezers. Spacers of unmodified thymidines (T) between biotin modified thymidines (BT) were designed to allow the different biotins to reach and bind different streptavidin molecules of a streptavidin bead. The HindIII-compatible overhang of Adaptor 1 contains a 5′ phosphate to allow ligation to DNA ends. Adaptor 1 and 2 are annealed using a mix containing 10 μM of each oligonucleotide in buffer 10 mM Tris-HCl 7.5, 50 mM NaCl. The mix is incubated at 85° C. for 30 seconds and cooled down at a ramp rate of 0.1° C./second using SimpliAmp thermal cycler (Applied Biosystems™ A24811).

Non-Directional Tandem Ligation with Biotin DNA Adaptor

To biotinylate the DNA ends of NTL products, 0.5 μM of BDA was added to NTL after 1 minute reaction. To assess the ligation of BDA to the DNA ends and consequent termination of ligation, DNA products were compared with NTL quenched after 3 minutes with 35 mM EDTA by agarose gel electrophoresis (FIG. 5A). NTL supplemented with BDA shows DNA bands corresponding to short DNA molecules that disappear after 3 minutes reaction, indicating that BDA stops ligation by capping the DNA ends with biotin. Successively, lower concentrations of BDA (25 nM, 62.5 nM and 125 nM) were tested resulting in blocking NTL at a concentration as low as 25 nM (FIG. 5B). Considering an initial concentration of 57.4 nM of linearized pUC19 at the start of NTL (114.8 nM of DNA ends) and assuming that a 2 to 1 ratio of BDA to DNA ends is sufficient to block the reaction, we speculate that after 1 minute only 12.5 nM of non-ligated DNA ends are present in solution. This further support our model according to which 1 minute of NTL marks the end of the linear phase with ˜90% of DNA ends ligated (see FIG. 2C).

### Analysis of Biotinylated NTL Products in Optical Tweezers

NTL reaction biotinylated with BDA is purified with Amicon™ Ultra-0.5 Centrifugal Filter Units (Merck, UFC503008) in buffer Tris-EDTA following manufacturer's instructions. Using optical tweezers experiments on C-Trap® (LUMICKS), biotinylated DNA molecules were tethered between two streptavidin-coated polystyrene beads, 1.76 μm in diameter (LUMICKS). Using standard LUMICKS' protocols, high efficiency of tethering is observed when NTL products are biotinylated within 1 minute reaction. Force distance curves were fitted with the inverted Odijk model from the python package Pylake to determine the contour length and therefore the number of repeats of each tether using the so-called extensible worm-like-chain model (Odijk, T. (1995). Stiff Chains and Filaments under Tension. Macromolecules 28, 7016-7018 (https://doi.org/10.1021/ma00124a044])) and (Wang M. D., Yin H., Landick R., Gelles J., Block S. M. (1997). Stretching DNA with optical tweezers., Biophysical journal (72, 1335-46) (https://doi.org/10.1016/S0006-3495(97)78780-0)).Tether length analysis showed a mixed population of DNA molecules with number of repeats ranging between 9 and 23 repeats with number of repeats with 33% of the tethers containing more than 16 repeats. Importantly, efficiency of tethering dramatically drops when biotinylation was performed in NTL after more than 1 minutes. Tethering is not observed when biotinylation is attempted with DNA molecules of NTL when the biotinylated adapters are added after more than 3 minutes confirming a 1 minute long of linear phase of NTL.

### Example 2: Directional Tandem Ligation

### PCR and Bsal Digestion of DNA Ends.

Directional tandem ligation reactions were performed either using a 2.079 kb DNA unit (2 kb) or a 3.065 kb DNA unit (3 kb). The 2 kb unit was produced by PCR using primers (Table 1, Fw-primer 3, and Rv-primer 4) to amplify region 33362 bp-35408 bp of Lambda DNA. The 3 kb unit was produced by PCR using primers (Table 1, Fw-primer 3, and Rv-primer 5) to amplify region 33362 bp-36394 bp of Lambda DNA. In addition to annealing sequences complementary to the sequences of Lambda DNA (Table 1, sequences in bold in Fw-primer 3, Rv-primer 4 and 5), these primers contain sequences that form Bsal restriction sites upon PCR reaction (Table 1, underlined in Fw-primer 3, Rv-primer 4 and 5). Thus, upon completion of the PCR, 2 kb and 3 kb DNA units were purified with Promega Kit (Wizard® SV Gel and PCR Clean-Up System, A9281) and digested with Bsal restriction enzyme (Bsal-HF®v2, NEB, R3733) according to manufacturers' instructions. Then, digested DNA was purified again with DNA purification Promega Kit according to the PCR clean-up protocol and then concentrated to either 200 ng/ul (2 kb) or 450 ng/ul (3 kb). By design, Bsal digestion of PCR products led to the formation of overhangs suitable for unidirectional intermolecular ligation (FIG. 2B). Importantly, these overhangs will still allow intramolecular ligation with formation of circular DNA molecule, therefore the labelling of DNA ends was performed at the end of the linear phase as described in NTL.

### Assembling Digoxigenin DNA Adaptor (DDA) and Biotin DNA Adaptor 2 (BDA_2)

DDA is formed upon annealing of two oligonucleotides (Table 1, Adaptor 3 and 4, and FIG. 2B) and is constituted by a 21 bp double stranded DNA formed by the complementary sequences of Adaptor 3 and 4 (Table 1, sequences in bold), flanked on one end with a 5′ overhang containing 3 digoxigenin moieties of the Adaptor 3 (Table 1, underlined sequence) and on the other end with a 5′ overhang of Adaptor 4 (Table 1, underlined sequence) complementary to the 5′ overhang generated by the Bsal digestion of the 5′ of either 2 kb unit or 3 kb unit (Table 1, underlined sequence and FIG. 2B). The digoxigenin moiety attached to the 5′ of Adaptor 3 blocks ligation reaction and allow binding to the anti-digoxigenin bead, while the additional 2 digoxigenin moieties attached internally to thymidines of the sequence are necessary to form additional binding sites with the anti-digoxigenin bead and stabilizes the tethers in optical tweezers. The BDA_2 is formed upon annealing of two oligonucleotides (Table 1, Adaptor 5 and 6, and FIG. 2B) and is constituted by a 24 bp double stranded DNA formed by the complementary sequences of Adaptor 5 and 6 (Table 1, sequences in bold), having on one end the 5′ modified with the 3 biotins of the Adaptor 5 (Table 1, 3×Biotin) and on the other end with a 5′ overhang of Adaptor 6 (Table 1, underlined sequence) complementary to the 5′ overhang generated by the Bsal digestion of the 3′ of either the 2 kb unit or the 3 kb unit (Table 1, underlined sequence and FIG. 2B). The 3 biotins attached to each other with one biotin attached to the 5′ of BDA_2 block the ligation reaction. The annealing procedures of both Adaptor 3 with Adaptor 4 to form DDA and Adaptor 5 with Adaptor 6 to form BDA_2 are performed similar to the annealing procedure of BDA in NTL by mixing 10 uM of each oligonucleotide in buffer 10 mM Tris-HCl 7.5, 50 mM NaCl. Then, the mix is incubated at 85° C. for 30 seconds and cooled down at a ramp rate of 0.1° C./second using SimpliAmp thermal cycler (Applied Biosystems™ A24811).

Directional Tandem Ligation of DNA with Biotin DNA Adaptors (BDA_2) and Digoxigenin DNA Adaptors (DDA)

DTL reactions were performed using 0.25 U/ul of Ligase T4 (Thermo Scientific™, 5 U/μL, EL001) mixed together with either 200 ng/ul of 2 kb units (150 nM) or 450 ng/ul (175 nM) of 3 kb units. DTL reactions and time courses were performed as described for NTL reactions. Like NTL, preliminary tests including time courses and biotinylation of DTL products indicated that the linear phase lasts 1 minute (data not shown and FIG. 1C). To distinguish the 5′ DNA end from the 3′ DNA end of DTL products, the 5′ DNA end was labelled with a digoxigenin DNA adaptor (DDA), while the 3′ DNA end with a biotin DNA adaptor (BDA_2) (see FIG. 2B).

After one minute of DTL, 1 μM of DDA and BDA_2 adaptors (see below) were added allowing the ligation of DDA and BDA_2 to the 5′ end and to the 3′ end, respectively, of either Bsal digested 2 kb unit or 3 kb unit. The Digoxigenin moiety attached to the 5′ of DDA and 3 biotins attached to the 5′ of BDA_2 block the DNA ends for further ligation. The resulting labelled DNA with multiple repeats is now suitable for tethering in optical tweezers in a dumbbell configuration where an anti-digoxigenin bead is in one optical trap and a streptavidin bead is in the other optical trap (FIG. 2B). The specificity of DDA for the 5′ sticky end and BDA_2 for the 3′ sticky end of DTL products allows deterministic tethering in a 5′-3′ direction from the anti-digoxigenin bead to the streptavidin bead.

Labelling with ATTO647N Shows Unidirectionality of Tandem Ligation.

To test the unidirectionality of DTL, we aimed to incorporate ATTO647N fluorophores asymmetrically in the DNA repeats. The region 33362 bp-35408 bp and 33362 bp-36394 bp of Lambda DNA contain two Nt.BstNBI restriction sites, one at position 33770 bp and the other at position 33783 bp (Kong et al. 2017 supra; Khun et al. 2008 (Kuhn, H., & Frank-Kamenetskii, M. D. (2008). Labeling of unique sequences in double-stranded DNA at sites of vicinal nicks generated by nicking endonucleases. Nucleic Acids Research, 36(7), 1-10 (https://doi.org/10.1093/nar/gkn107)). These Nt.BstNBI sites correspond to positions 425 bp and 438 bp of both 2 kb and 3 kb units. Digestion of DTL products by Nt.BstNBI forms two nicks at a distance of 13 bp from each other. This allows labelling of DTL products using 13 nt oligonucleotide containing a ATTO647N modified T (Table 1, ATTO647N-13 nt) essentially as previously described (Kong et al. 2017; Khun et al. 2008). Briefly, DTL was digested with Nt.BstNBI (NEB, R0607), mixed with a 50-fold excess of ATTO647N with respect to DNA repeats, incubated at 55° C. for 5 minutes and finally cooled down to 22° C. at a ramp rate of 0.2° C./s. Then, the mix is purified using Amicon™ Ultra-0.5 Centrifugal Filter Units (Merck, UFC503008) and nicks are solved using T4 DNA Ligase (Thermo Scientific™, 5 U/μL, EL001). In this way, ATTO647N fluorophores were incorporated at 429 bp from the 5′ end of both 2 kb and 3 kb repeats, while either at 1626 bp from the 3′ end of 2 kb repeats or at 2612 bp from the 3′ end of 3 kb repeats. Therefore, unidirectionality of DTL should lead to equidistant ATTO647N in DTL products, while a random direction ligation would lead to two different distances between ATTO647N fluorophores in DNA repeats. Fluorescence imaging of DTL DNA tandem repeats on C-trap® (LUMICKS) showed that ATTO647N fluorophores are equally distant in both 2 kb repeats (data not shown) and 3 kb repeats (FIG. 7A).

### Analysis of the DTL Products in Optical Tweezers

Biotinylated and digoxigenin-labelled DTL products are purified with Amicon™ Ultra-0.5 Centrifugal Filter Units (Merck, UFC503008) in buffer Tris-EDTA following manufacturer's instructions. Using optical tweezers experiments on C-trap® (LUMICKS), DNA molecules are tethered to a 2.06 μm anti-digoxigenin bead (LUMICKS) and to a 1.36 μm streptavidin-coated bead (LUMICKS). Force distance curves were fitted with the inverted Odijk model from the python package Pylake to determine the contour length and therefore the number of repeats of each tether using the so-called extensible worm-like-chain model (Odijk, T. (1995). Stiff Chains and Filaments under Tension. Macromolecules 28, 7016-7018 (https://doi.org/10.1021/ma00124a044)) and (Wang M. D., Yin H., Landick R., Gelles J., Block S. M. (1997). Stretching DNA with optical tweezers., Biophysical journal (72, 1335-46) (https://doi.org/10.1016/S0006-3495(97)78780-0)). Tether length analysis showed a mixed population of DNA molecules in DTL with either 2 kb or 3 kb repeats. In both cases, more than 60% of the tethers contains more than 7 DNA repeats (data not shown and FIG. 7B).

1. A method for producing linear DNA molecules with two or more repeating units of a target DNA sequence, the method comprising steps of:
a) providing a plurality of the target DNA sequence and a DNA ligase and subjecting the plurality of target DNA sequence to a ligation reaction to obtain a first mixture of DNA molecules varying in length and number of repeats, wherein each DNA molecule in the first mixture of DNA molecules comprises at least two or more repeating units of the target DNA sequence, and
b) stopping the ligation reaction at an end of a linear phase of the ligation reaction, and
c) recovering said first mixture of DNA molecules.

2. The method according to claim 1, further comprising a step of:
d) subjecting the first mixture of DNA molecules obtained in step c) to a second ligation reaction in a presence of a DNA ligase and DNA adaptor molecules to obtain a second mixture of DNA molecules varying in length and number of repeats, wherein each DNA molecule in the second mixture of DNA molecules comprises at least two or more repeating units of the target DNA sequence, and part of the DNA molecules in the second mixture of DNA molecules consists of adapted linear DNA molecules.

3. A method for producing linear DNA molecules with two or more repeating units of a target DNA sequence, the method comprising steps of:
a) providing a plurality of the target DNA sequence and a DNA ligase and subjecting the target DNA sequence to a ligation reaction to obtain a first mixture of DNA molecules varying in length and number of repeats, wherein each DNA molecule in the first mixture comprises at least two or more repeating units of the target DNA sequence,
b) adding DNA adaptor molecules to the first mixture of DNA molecules at an end of a linear phase of the ligation reaction to obtain a second mixture of DNA molecules varying in length and number of repeats, wherein each DNA molecule in the second mixture of DNA molecules comprises at least two or more repeating units of the target DNA sequence, and part of the DNA molecules in the second mixture consists of adapted linear DNA molecules.

4. The method according to claim 2, wherein the second mixture of DNA molecules is further subjected to a functionalization step wherein ends of the adapted linear DNA molecules are functionalized by labeling the ends of the adapted linear DNA molecules with a functional moiety to obtain a third mixture of DNA molecules varying in length and number of repeats, wherein each DNA molecule in the third mixture of DNA molecules comprises at least two or more repeating units of the target DNA sequence, and part of the DNA molecules in the third mixture consists of functionalized linear DNA molecules.

5. The method according to claim 2, wherein the DNA adaptor molecules have been functionalized by labeling with a functional moiety prior to addition to the first mixture of DNA molecules, to produce the second mixture of DNA molecules varying in length and number of repeats, wherein each DNA molecule in the second mixture of DNA molecules comprises at least two or more repeating units of the target DNA sequence, and part of the DNA molecules in the second mixture of DNA molecules are functionalized linear DNA molecules.

6. The method according to claim 4, wherein the functional moiety is half of a conjugation pair.

7. The method according to claim 4, wherein the functional moiety is selected from the group consisting of biotin and digoxigenin.

8. The method according to claim 1, wherein the target DNA sequence has a size in a range of and including 400 to 5000 base pairs.

9. The method according to claim 1, wherein the target DNA sequence has been modified to comprise one or more lesions, one or more fluorescent labels, or any combination thereof.

10. The method according to claim 1, wherein the DNA adaptor molecules are added to the first mixture of DNA molecules in at least a two-fold excess of a concentration of DNA in the first mixture of DNA molecules.

11. A mixture of DNA molecules obtained by the method according to claim 1.

12. A composition comprising a mixture of DNA molecules varying in length and number of repeats, wherein each DNA molecule comprises at least two or more repeating units of a target DNA sequence, and part of the DNA molecules consists of functionalized linear DNA molecules.

13. The composition according to claim 12, wherein the composition comprises at least 5% by weight of the functionalized linear DNA molecules, based on a total weight of the DNA molecules.

14. A method of using the mixture of DNA molecules obtained by the method according to claim 4 or the composition according to claim 12 in a single-molecule assay comprising a step of conducting the single molecule assay in an assay composition comprising the mixture of DNA molecules obtained by the method according to claim 4.

15. A single-molecule assay comprising a step of
a) providing a mixture of DNA molecules according to claim 4 or a composition according to claim 12 as a DNA substrate, and beads that have been functionalized with groups that are capable of binding specifically to the functional moiety of the functionalized linear DNA molecules in said mixture or composition,
b) bringing said mixture or composition into contact with said beads to form linear DNA molecules trapped between two or more of said beads,
c) subjecting the linear DNA molecules trapped between the two or more beads to distance analysis to select the linear DNA molecules that have a predetermined length, and
d) measuring interaction of molecules with the selected linear DNA molecules.

16. The method according to claim 3, wherein the second mixture of DNA molecules is further subjected to a functionalization step wherein ends of the adapted linear DNA molecules are functionalized by labeling the ends of the adapted linear DNA molecules with a functional moiety to obtain a third mixture of DNA molecules varying in length and number of repeats, wherein each DNA molecule in the third mixture of DNA molecules comprises at least two or more repeating units of the target DNA sequence, and part of the DNA molecules in the third mixture consists of functionalized linear DNA molecules.

17. The method according to claim 3, wherein the DNA adaptor molecules have been functionalized by labeling with a functional moiety prior to addition to the first mixture of DNA molecules, to produce the second mixture of DNA molecules varying in length and number of repeats, wherein each DNA molecule in the second mixture of DNA molecules comprises at least two or more repeating units of the target DNA sequence, and part of the DNA molecules in the second mixture of DNA molecules are functionalized linear DNA molecules.

18. The method according to claim 5, wherein the functional moiety is half of a conjugation pair.

19. The method according to claim 5, wherein the functional moiety is selected from the group consisting of biotin and digoxigenin.

20. The method of claim 14, wherein the single molecule assay is selected from the group consisting of a flow stretching assay, a TIRF assay, and an optical tweezers assay.
